(19) United States
(12) Patent Application Publication
(10) Pub. No.: US 2023/0001077 A1
(43) Pub. Date: Jan. 5, 2023

(54) SYRINGE BRACKET WITH A SEAL

(71) Applicant: B. Braun Melsungen AG, Melsungen (DE)

(72) Inventors: Jan Schwarz, Melsungen (DE); Marcel Stich, Nieste (DE); Matthias Schwalm, Schwalmstadt (DE); Joachim Schuetz, Fulda (DE); Norbert Koop, Rellingen (DE); Jan Sokoll, Nortorf (DE); Stefan Espenhahn, Hamburg (DE)

(21) Appl. No.: 17/851,119

(22) Filed: Jun. 28, 2022

(30) Foreign Application Priority Data
Jul. 1, 2021 (DE) 20 2021 103 509.9

(51) Int. Cl. A61M 5/145

(52) U.S. Cl. CPC A61M 5/1458; A61M 2005/3143

(57) ABSTRACT

A syringe pump includes a syringe bracket fixing a syringe in a syringe channel. The syringe bracket is connected to the syringe pump via a tubular bracket arm and can be pulled out of the syringe pump and locked in the pulled-out position by a catch mechanism so as to insert a syringe into the syringe pump, and after the catch mechanism is released, a spring preload on the bracket arm pulls the syringe bracket back to the syringe pump. The retraction of the bracket arm is damped by a motion damper.

### CROSS-REFERENCE TO RELATED APPLICATION

This application claims priority under 35 U.S.C. § 119 to German Application No. 20 2021 103 509.9, filed Jul. 1, 2021, the content of which is incorporated by reference herein in its entirety.

### FIELD

The disclosure relates to a syringe pump with a syringe bracket for fastening a syringe in the syringe pump.

### BACKGROUND

Syringe pumps are used in medicine to deliver to patients a defined dose of medication from a syringe. The syringe is automatically squeezed at a specific rate to deliver a defined amount of medication to a patient over a defined time. Syringe pumps are known, for example, from EP 0 566 825 A1. DE 202 00 885 U1 discloses a syringe pump with a plunger brake. The plunger brake is mounted in a syringe bracket. The plunger brake fastens the plunger of a syringe after insertion. To enable the syringe to be squeezed, the plunger plate at the end of the syringe is gripped by a drive head with claws. To enable the claws to grip the plunger plate, the plunger of the syringe must be fixed. The plunger brake presses the piston against a contact surface and fixes it on the contact surface. The plunger brake is located in the syringe bracket, which is pulled out of the syringe pump for inserting the syringe. The syringe bracket is rotated by 90° about its axis and is locked in the rotated position. The syringe is inserted and the syringe bracket is released from the lock. As the syringe bracket is pre-tensioned by a spring, it is pulled towards the syringe. When the spring is pre-tensioned too much, the syringe yoke will recoil. This is unpleasant for a user of the syringe pump. Furthermore, damage can occur to the syringe and/or the syringe pump if the syringe is pulled back too far.

### SUMMARY

The disclosure is based on the object of providing a syringe pump including a syringe bracket, by which pleasant and high-quality closure of the syringe bracket is ensured.

The disclosure relates to a syringe pump including a syringe bracket that fixes a syringe in a position, the syringe bracket being connected to the syringe pump via a tubular bracket arm and can be pulled out of the syringe pump and locked in the pulled-out position by means of a catch mechanism so as to insert a syringe into the syringe pump. After the catch mechanism is released, a spring preload on the bracket arm pulls back the syringe bracket to the syringe pump. According to the disclosure, retraction of the bracket arm is damped by a motion damper.

A high spring force makes undamped retraction of the syringe bracket unpleasant to a user and reduces the impression of quality and good processing of the syringe pump. Furthermore, the syringe bracket hits its contact surface at high speed, resulting in a loud noise. The impact can also result in damage to the syringe pump and/or the syringe bracket. Therefore, the rapid movement of the syringe bracket towards the syringe pump is dampened. The damping causes the syringe bracket to retract gently. There is no noise when the syringe bracket hits its stop position. Springing back of the syringe bracket is gently cushioned by damping. Thus, to a user, the gentle retraction of the syringe bracket provides a user-friendly overall impression.

According to another feature of the disclosure, the tubular bracket arm pierces the housing of the syringe pump at an aperture/a hole and has a seal at the aperture. The bracket arm connects the syringe pump and the syringe bracket. The bracket arm is fastened inside the syringe pump and protrudes through the aperture from the housing of the syringe pump. The aperture is sealed by a seal. Due to the seal, dust or liquid cannot enter the housing. The seal fulfills at least protection type IP44 and is implemented as a round, elastic seal.

According to a further feature of the disclosure, the seal is a motion damper. The seal is in contact with the bracket arm and friction is provided between the bracket arm and the seal. The seal presses a tube of the bracket arm, i.e. the seal exerts a contact pressure onto the tube of the bracket arm. When the bracket arm is moved in an axial direction friction occurs between the seal and the tube. Friction obstructs an axial movement of the bracket arm. Thus, friction also obstructs retraction of the bracket arm and thus dampens retraction of the bracket arm.

According to a further feature of the disclosure, the outer diameter of the tubular bracket arm increases in a longitudinal direction of the bracket arm towards the syringe bracket. Hence, when the bracket arm is pulled into the housing in an axial direction, the outer diameter of the tube, which is pulled through the hole and the seal, increases. Correspondingly, the outer diameter decreases when the tube is pulled out of the housing.

According to another feature of the disclosure, the friction between the seal and the bracket arm increases with increasing diameter of the bracket arm. Due to the increasing outer diameter of the tube, the contact pressure of the seal on the tube increases, resulting in an increase of friction between the seal and the bracket arm. Thus, the friction increases when the bracket arm is pulled into the housing.

According to another feature of the disclosure, the bracket arm is connected to the syringe pump housing by a spring on a side opposite to the syringe bracket. The spring is deflected when the syringe bracket is outwardly pulled out of the housing. When the syringe bracket is released, the spring pulls the bracket arm together with the syringe bracket towards the wall of the housing. By this, the syringe bracket is pulled into the housing. The motion of the syringe bracket into the housing is dampened by the seal.

According to another feature of the disclosure, damping increases approximately exponentially in relation to a decreasing deflection of the syringe bracket. Since the friction force increases with decreasing deflection of the bracket arm and the spring force decreases with decreasing deflection of the spring and thus also with decreasing deflection of the bracket arm, damping increases progressively with decreasing deflection of the bracket arm. As the two effects of a decreasing spring force and increasing friction add up, the damping increases approximately in an exponential function.

According to a further feature of the disclosure, the increasing friction progressively dampens recoil of the bracket arm in the direction of the syringe pump. By overlapping of the effects described above, the damping increases the further the bracket arm is pulled into the housing.

According to a further feature of the disclosure, the increasing diameter of the bracket arm is a draft angle. The tube of the bracket arm is cast. Since cast parts must be demolded, cast parts have a draft angle for easier demolding. In the tube of the bracket arm, the draft angle runs such that the diameter of the tube becomes larger towards the syringe bracket. This is desirable for the damping described above. In this way, the tube does not have to be reworked in a costly manner, but the desired bevel/diameter change already results from the manufacturing process.

According to another feature of the disclosure, the syringe bracket retracts with a soft-close mechanism. A soft-close mechanism is known, for example, from drawers. A user merely needs to tap the open drawer and the drawer closes gently by itself. Retraction of the syringe bracket works in a similar way. All the user has to do is to release the locking mechanism and the syringe bracket will gently retract to the syringe pump housing by itself. The bracket arm retracts into the housing.

### BRIEF DESCRIPTION OF THE DRAWING FIGURES

FIG. 1 shows an isometric view of a syringe pump;

FIG. 2 shows a syringe pump including an inserted syringe;

FIG. 3 shows a syringe bracket in the syringe pump;

FIG. 4 shows a plunger brake in the syringe bracket;

FIG. 5 is a longitudinal section through the bracket arm of the syringe bracket and a syringe pump housing; and

FIG. 6 is a diagram showing interaction of the friction of the seal and the tensile force of a spring.

### DETAILED DESCRIPTION

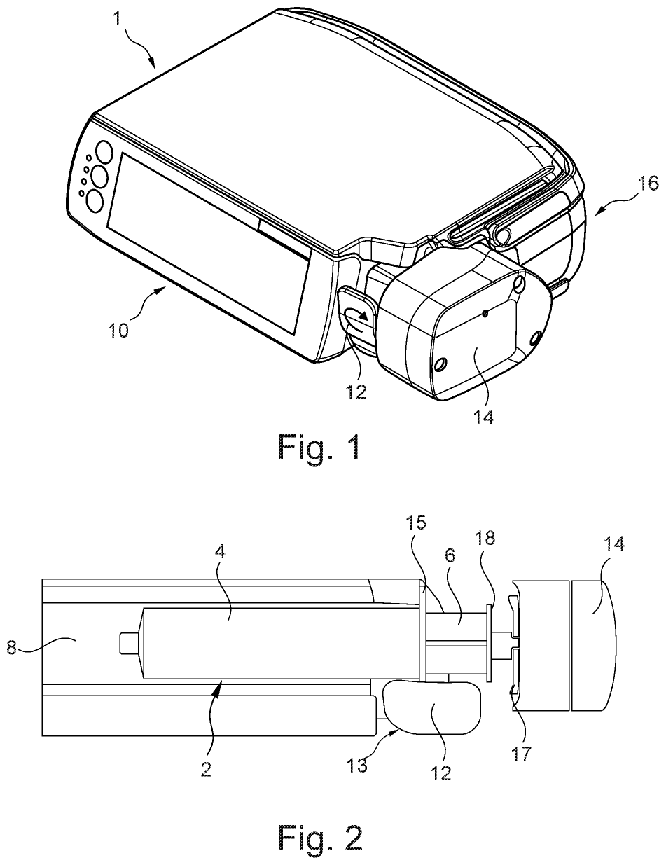

FIG. 1 shows a syringe pump 1. The basic mode of operation of syringe pump 1 is sufficiently described in EP 0 566 825 A1, so that a general description of syringe pump 1 herein is dispensed with.

The syringe pump 1 serves for squeezing a syringe 2 having a syringe cylinder 4 and a plunger 6. The syringe 2 is inserted in the syringe pump 1 in a syringe channel 8. For this, a front flap 10 of the syringe pump 1 is lifted up downwards in order to release the syringe channel 8. Moreover, a syringe bracket 12 fixing the plunger is pulled out of the syringe pump 1. The syringe bracket 12 is pulled out of syringe pump 1 and can be rotated by 90°, so as to be caught in the pulled-out position in a catch 13.

A drive head 14 extends laterally out of a pump housing 16 of syringe pump 1 on the outside. The syringe 2 can then be inserted into the syringe pump 1. During operation of the syringe pump 1, the drive head 14 presses the plunger 6 into the syringe cylinder 4 and thus empties the syringe 2. A syringe pump 1 with inserted syringe 2 is shown in FIG. 2. The drive head 14 is extended and the syringe bracket 12 is folded down and locked in place.

After inserting the syringe 2, the front flap 10 is folded in. The syringe 2 is fastened in the syringe pump. For this, the syringe 2, on the one hand, lies in syringe channel 8. On the other hand, the syringe wings 15 of syringe cylinder 4 are held on the pump side by an axial fixation (not shown) in pump housing 16, and on the side of the syringe bracket by a fixing on syringe bracket 12. The drive head 14 has clamps 17 encompassing the plunger plates 18 of syringe 2. For this purpose, the drive head is moved towards the plunger plates 18. To enable the clamps 17 to grip the plunger plates 18, the plunger 6 of the syringe 2 must be fixed. A plunger brake 20 fixes the plunger 6 to a contact face 22 (as shown in FIG. 4). To do this, after inserting the syringe 2, the syringe bracket 12 is released from catch mechanism 13. The syringe bracket 12 is pressed against the syringe through the spring load of a spring 24. Moreover, the plunger brake 20 moves out of the syringe bracket 12 and presses the plunger 6 against contact surface 22, thereby fixing the plunger 6. The plunger brake 20 is known from DE 202 00 885 U1. The combination of a syringe channel 8, an axial fixation of the syringe wings 15 and a plunger brake 20 fixes the syringe 2 in the syringe pump 1. The mode of operation of the syringe yoke bracket is explained below.

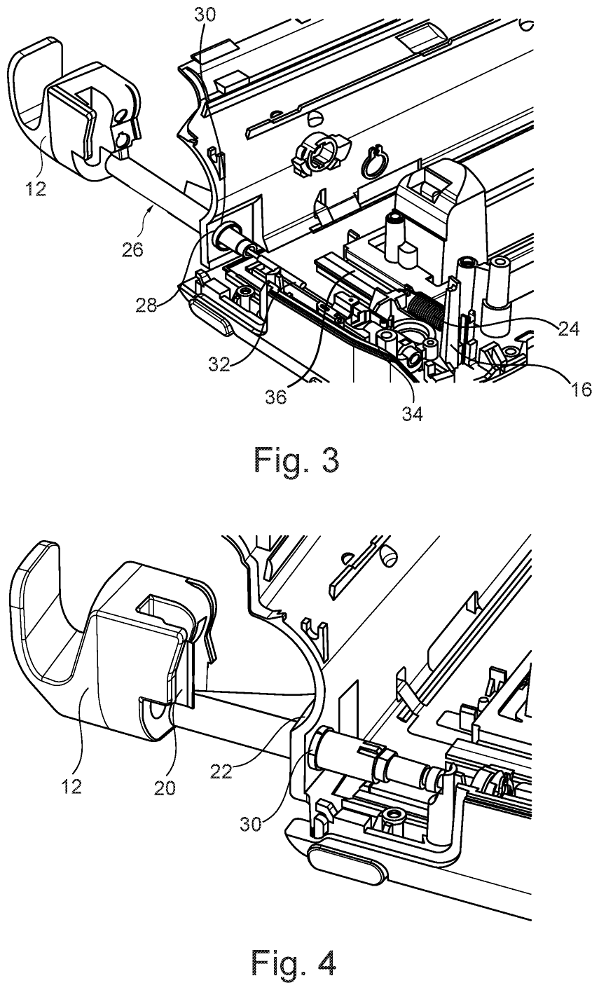

FIG. 3 shows the syringe yoke 12 in the pump housing 16 of syringe pump 1. The side wall of syringe pump 1 and further components of syringe pump 1 were omitted in this view in order to make the syringe bracket 12 visible. The syringe bracket 12 is positioned on a side of syringe 2 facing away from syringe pump 1 and protrudes from pump housing 16. The syringe bracket 12 is connected to syringe pump 1 via a bracket arm 26. The bracket arm 26 passes below the syringe 2 and is aligned orthogonally to the longitudinal direction of the syringe 2. The bracket arm 26 pierces through the pump housing 16 in a hole 28. A seal 30 is fastened between the hole 28 and the bracket arm 26. The seal 30 is in contact with an outer side of the bracket arm 12 and seals the hole 28 against the penetration of contaminants or liquids.

On the side of bracket arm 26 opposite the syringe bracket 12, the bracket arm 26 is connected to a carriage 34, which is to be moved in the longitudinal direction of the bracket arm 26. A drive element of plunger brake 20, for example, an electric motor (not shown), is fixed to the carriage 34. The motor is connected to a pull rod 32 by a thread (not shown), which is moved in an axial direction by the motor. The pull rod 32 runs inside the tubular bracket arm 26 and activates the plunger brake 20 in the syringe bracket 12. The pull rod 32 thus forms a linear drive activating the plunger brake 20. The carriage 34 with the motor is connected to the bracket arm 26 and thus to the syringe bracket 12 by a driver and thus can be moved with the syringe bracket 12. For this purpose, the carriage is supported in a guide 36, which allows a movement in the longitudinal direction of the bracket arm 26, which is transverse to the longitudinal direction of the syringe 2. The carriage 34 is connected to the pump housing 16 by a spring 24, i.e. when the syringe bracket 12 is pulled out of the pump housing 16, the carriage 34 moves along and moves away from the pump housing 16 with which it is connected through the spring 24. This causes the spring 24 to be tensioned. When the syringe bracket 12 is released, the carriage 34 is pulled back to the docking point of the spring 24, and thus also the syringe bracket 12 is pulled back into the pump housing 16. The plunger brake 20 in an extended state is shown in FIG. 4.

When the syringe 2 is taken from the syringe pump 1, the syringe bracket 12 is extracted from the syringe pump 1, as in the case of inserting the syringe 2, is rotated by 90° about its axis, and is locked in place. Without the syringe 1, the path from the locking mechanism 13 to the stop point, which now is no longer the syringe 2, but the contact surface 22 of the syringe pump 1, is longer. Since the syringe bracket 12 is spring-loaded, the syringe bracket 12 would spring back/snap back in the direction of the syringe pump 1. This is not ergonomic for a user and makes a loud noise. The rapid movement and the impact could damage the syringe bracket 12 and/or another component of the syringe pump 1.

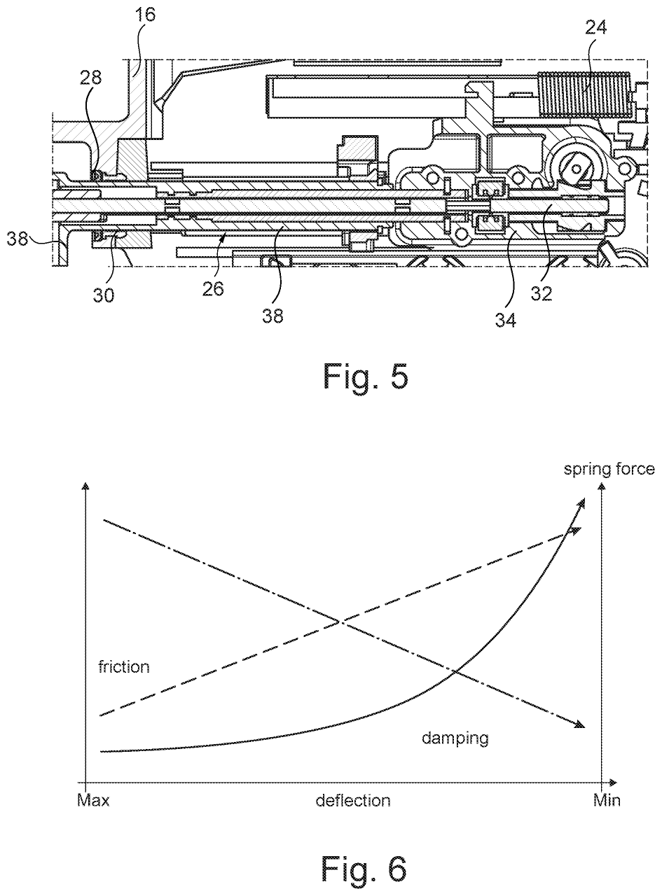

For this reason, the bracket arm 26 has a damping device. The functioning of the damping is shown in FIG. 5. FIG. 5 shows a longitudinal section through the bracket arm 26 and the pump housing 16. The bracket arm 26 substantially is an elongated tube 38. The pull rod 32 of the linear drive of plunger brake 20 extends in the tube 38. In the hole 28 in the pump housing 16 through which the tube 38 runs, the round seal 30 is seated. The seal 30 is made of an elastic material and is in contact with the tube 28 of bracket arm 12, thus creating friction between the seal 30 and the outer surface of the tube 38. The diameter of the tube 38 decreases in the longitudinal direction. On the side of the tube 38 that is closer to the syringe bracket 12, the tube 38 has the largest outer diameter. On the opposite side closer to the spring 24, the tube 38 has the smallest outer diameter. Due to an increasing outer diameter in the longitudinal direction of the bracket arm 12, also the friction between the seal 30 and the tube 38 increases depending on the position, i.e., when the syringe bracket 12 is pushed/drawn into the pump housing 16 in the direction of the syringe pump 1, friction between the tube 38 and the seal 30 continues to increase in a constant manner the further the tube 38 is pushed into the pump housing 16. The spring has a constant force-displacement curve, i.e. the tensile force 24 of spring 24 decreases in a constant manner as the deflection of the spring 24 decreases. The interaction of the spring force and the friction force of the seal 30 results in damping the syringe bracket 12.

FIG. 6 shows this relationship in a displacement-force diagram. The spring force, which is characterized by a dashed line, decreases as the deflection of spring 24 decreases. The farther the bracket arm 26 is pulled into the pump housing 16 by the spring 24, the stronger the friction force between the seal 30 and the tube 38 becomes. The friction is represented by a dotted dashed line and increases in a constant manner with the deflection. Since the two effects overlap and since both effects influence damping (continuous line in FIG. 6) of the syringe bracket 12, damping increases depending on the position approximately in an exponential curve. The damping increases the farther the bracket arm 26 is pulled into the pump housing 16. Thus, damping is increasing progressively. Thus, the syringe bracket 12 gently strikes its stop. Due to the progressive damping, a “soft-close mechanism” is realized.

1. A syringe pump comprising:
a housing having a syringe channel; and
a syringe bracket for fixing a syringe in the syringe channel,
the syringe bracket connected to the housing via a bracket arm that is tubular,
the bracket arm configured to be pulled out of the syringe pump and locked in a pulled-out position by a catch mechanism to facilitate insertion of a syringe into the syringe pump,
the bracket arm comprising a spring preload configured to pull the syringe bracket back into the housing after the catch mechanism is released,
the syringe pump further comprising a motion damper to dampen retraction of the bracket arm.

2. The syringe pump according to claim 1, wherein the bracket arm pierces the housing of the syringe pump at an aperture and has a seal at the aperture.

3. The syringe pump according to claim 1, wherein the motion damper is a seal.

4. The syringe pump according to claim 3, wherein the seal is in contact with the bracket arm and wherein friction occurs between the bracket arm and the seal.

5. The syringe pump according to claim 1, wherein the bracket arm is connected to the pump housing by a spring on a side opposite to the syringe bracket.

6. The syringe pump according to claim 1, wherein an outer diameter of the bracket arm increases towards the syringe bracket in a longitudinal direction.

7. The syringe pump according to claim 6, wherein a friction between the motion damper and the bracket arm increases along the bracket arm as the outer diameter of the bracket arm increases.

8. The syringe pump according to claim 7, wherein the friction progressively dampens a retraction of the bracket arm in the direction of the syringe pump.

9. The syringe pump according to claim 8, wherein dampening of the retraction increases approximately exponentially in relation to a decreasing deflection of the syringe bracket.

10. The syringe pump according to claim 6, wherein the outer diameter of the bracket arm increases along a draft angle.
